(19) United States
(12) Patent Application Publication
(10) Pub. No.: US 2026/0053990 A1
(43) Pub. Date: Feb. 26, 2026

(54) Wearable Lactation Aid

(71) Applicant: Merkat Inc, San Francisco, CA (US)

(72) Inventors: Merima Ramic, San Francisco, CA (US); Katarzyna Natalia Karauda, Belvedere Tiburon, CA (US)

(21) Appl. No.: 18/813,483

(22) Filed: Aug. 23, 2024

(51) Int. Cl. A61M 1/06

(52) U.S. Cl. CPC A61M 1/066; A61M 1/0697

(57) ABSTRACT

A wearable lactation aid apparatus and system with a controller that operates to express breast milk from a lactating user using compression pressure and draw the expressed breast milk from a reservoir into a collection container with suction. The apparatus includes a compression device positioned on a breast of the user. The compression device is configured to repeatedly apply and release compression on the breast. A nipple cone is positioned on the breast of the user and is connected to a shield. A pump connected to the shield and in fluid communication with a collection container positioned between the pump and the shield creates a vacuum in the collection container to draw in the expressed milk.

### BACKGROUND INFORMATION

### 1. Field

The present disclosure relates generally to breast pumps and, in particular, to a wearable device for expressing and collecting breast milk from a lactating user.

### 2. Background

Current breast pumps in the market face a variety of issues that significantly impact user satisfaction and efficiency. One of the primary problems is discomfort during use, as models have hard, rigid flanges that can cause pain and irritation to the breast and nipple area. This discomfort often leads to users not being able to pump as frequently or for as long as needed, reducing milk output and causing frustration. Additionally, many breast pumps are bulky and inconvenient to use, making them difficult to transport and operate discreetly, which is a significant concern for working mothers who need to pump in different environments.

Another common issue with existing breast pumps is their noise level. Many pumps are loud and disruptive, making it challenging for mothers to use them in shared or quiet spaces without drawing attention or causing disturbances. Furthermore, the suction mechanisms in many models are not adjustable enough to cater to the diverse needs of different users, leading to inefficiencies in milk extraction and added discomfort. Cleaning and maintaining breast pumps also pose a problem, as many models have multiple small parts that are difficult to sanitize thoroughly, increasing the risk of contamination and affecting the overall hygiene and safety of the pumping process. These issues highlight the need for innovative solutions that prioritize comfort, efficiency, and ease of use for breastfeeding mothers.

Therefore, it would be desirable to have a method and apparatus that takes into account at least some of the issues discussed above, as well as other possible issues.

### SUMMARY

An illustrative embodiment of the present disclosure provides a wearable lactation aid apparatus. The wearable lactation aid includes a nipple cone, a compression device, and a shield. The shield is connected to the nipple cone. The compression device is positioned between the shield and the nipple cone.

Another illustrative embodiment of the present disclosure provides a system for expressing and collecting breast milk from a lactating user. The system includes an inflatable bag positioned on a breast of the user. A pump is connected to the inflatable bag. The pump repeatedly inflates and deflates the inflatable bag to apply and release compression on the user's breast. A nipple cone is positioned on the breast of the user. The nipple cone extends through the inflatable bag for connection to a shield. The pump is connected to the shield and is in fluid communication with a collection container. The collection container is positioned between the pump and the shield.

A further illustrative embodiment of the present disclosure provides a method for expressing and collecting breast milk. A compression device positioned on the breast of a lactating user applies compression pressure to a breast of a lactating user to express breast milk from the breast. Expressed breast milk is collected with suction applied to a shield connected to the compression device. The expressed milk is collected in a collection container.

The features and functions can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 is an illustration of a block diagram of a lactation collection environment in accordance with an illustrative embodiment;

FIG. 2 is an illustration of a block diagram of a lactation aid apparatus in accordance with an illustrative embodiment;

FIG. 3 is an illustration of components of a housing and collection container of a lactation aid apparatus in accordance with an illustrative embodiment;

FIG. 4 is an illustration of components of a housing and collection container of a lactation aid apparatus in accordance with an illustrative embodiment;

FIG. 5 is an illustration of a deflated inflatable bag and a nipple cone of a lactation aid apparatus in accordance with an illustrative embodiment;

FIG. 6 is an illustration of an inflated inflatable bag and a nipple cone of a lactation aid apparatus in accordance with an illustrative embodiment;

FIGS. 7A-7B are illustrations of a pair of shields of a lactation aid apparatus in accordance with an illustrative embodiment;

FIG. 8 is an illustration of a shield and tubing connected to a nipple cone and inflatable bag of a lactation aid apparatus in accordance with an illustrative embodiment;

FIG. 9 is an illustration of a cross-sectional view of a shield, a nipple cone, and inflatable bag of a lactation aid apparatus in accordance with an illustrative embodiment; and

FIG. 10 is an illustration of a flowchart of a process for expressing and collecting breast milk in accordance with an illustrative embodiment.

### DETAILED DESCRIPTION

The illustrative embodiments recognize and take into account one or more different considerations. For example, the illustrative embodiments recognize and take into account that typical breast pumps may be uncomfortable to use incorporating hard, rigid materials that may cause pain and irritation to the user. The illustrative embodiments recognize and take into account that typical breast pumps apply suction to the breast of a user to encourage the breast to express breast milk. The illustrative embodiments recognize and take into account that the suction pressure required to stimulate breast milk expression may be painful and uncomfortable. The illustrative embodiments recognize and take into account that typical breast pumps are loud, bulky and difficult to operate discreetly. The illustrative embodiments recognize and take into account that the small parts of typical breast pumps are difficult to sanitize thoroughly.

Thus, the illustrative embodiments provide an apparatus, system, and method for expressing and collecting breast milk from a lactating user that is comfortable, efficient, and easy to use.

Further, the illustrative embodiments provide a wearable device for expressing and collecting breast milk that is hands-free, discreet, and portable. The illustrative embodiments provide a wearable device that can be concealed under traditional clothing. The illustrative embodiments provide a device that uses pain-free compression to stimulate production of breast milk and gentle, near silent suction to collect the extracted breast milk.

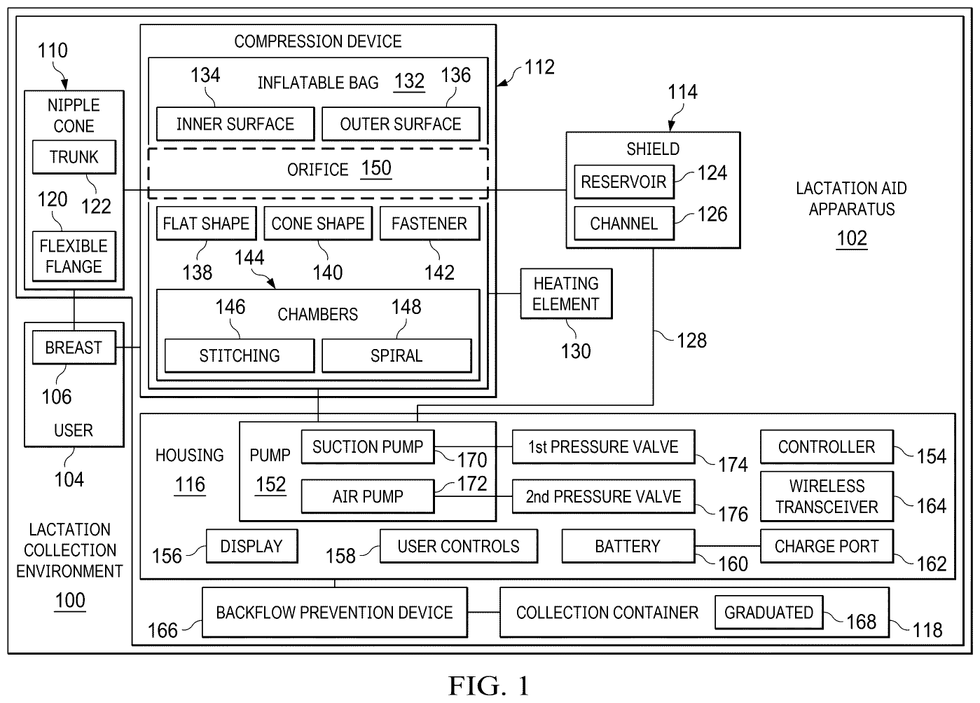

With reference now to the figures and, in particular, with reference to FIG. 1, an illustration of a block diagram of a lactation collection environment is depicted in accordance with an illustrative embodiment. In this illustrative example, lactation collection environment 100 includes lactation aid apparatus 102 capable of expressing breast milk from user 104 and collecting the expressed milk. User 104 includes breast 106. Breast 106 is a lactating breast. Lactation is the process of making and secreting milk from the mammary glands in the breasts. Lactation usually begins during pregnancy when changes in certain hormones signal the mammary glands to make milk.

In this illustrative example, lactation aid apparatus 102 includes nipple cone 110, compression device 112, shield 114, housing 116, and collection container 118.

Nipple cone 110 is flexible and is comprised of medical grade silicone. Nipple cone 110 includes flexible flange 120 and trunk 122. Trunk 122 is cylindrical shaped and extends from flexible flange 120. Flexible flange 120 is bow shaped, in other words, arcuate. The concave side of the arcuate shaped flexible flange 120 is sized and shaped to conform to breast 106. Nipple cone 110 surrounds the nipple of breast 106 and trunk 122 is axially aligned with the nipple of breast 106 of user 104.

Shield 114 is connected to nipple cone 110. Shield 114 is generally cone shaped to conform to the natural shape of breast 106. The concave side of shield 114 includes an internal shoulder that friction fits with trunk 122. In other words, shield 114 can be snapped onto and off of nipple cone 110 as normal use requires. Reservoir 124 is formed when nipple cone 110 is connected to shield 114 and flexible flange 120 of nipple cone 110 is fitted to user 104. When using lactation aid apparatus 102, breast milk expressed from user 104 may pool in reservoir 124 before the expressed breast milk is collected in collection container 118. In other words, reservoir 124 provides an initial collection point for expressed breast milk. Shield 114 includes channel 126. Channel 126 extends through shield 114. Channel 126 of shield 114 is connected to collection container 118 with tubing 128. Tubing 128 is comprised of medical grade silicone tubing. Channel 126 and tubing 128 provide fluid communication between reservoir 124 and collection container 118.

Compression device 112 is fitted to breast 106 of user 104. Compression device 112 repeatedly applies and releases compression pressure to breast 106. The compression pressure stimulates breast 106 to express breast milk. Since compression device 112 will directly contact breast 106 of user 104, compression device may include heating element 130. Heating element 130 can heat compression device 112 to a temperature that is comfortable for user 104.

Compression device 112 may provide compression pressure to breast 106. Compression device 112 may include, for example, pneumatic activated pads or panels, collapsible cones, inflatable airbags, etc. It's important to note, in contrast to breast pumps of the prior art that use uncomfortable and often painful suction to produce breast milk from a lactating user, lactation aid apparatus 102 incorporates gentle compression pressure applied by compression device 112 on the breast to stimulate the expression of breast milk. The manner of delivery of the compression pressure is secondary to the use of compression pressure itself.

In this illustrated example, compression device 112 may be inflatable bag 132. Inflatable bag 132 includes inner surface 134 and outer surface 136. Inner surface 134 is for contact with breast 106 of user 104. In use, flexible flange 120 abuts inner surface 134 and shield 114 abuts outer surface 136. Heating element 130 may be connected to inflatable bag 132 such that heating element can apply heat to inner surface 134. Heating element 130 may be removably connected to inflatable bag 132 at inner surface 134.

Inflatable bag 132 includes flat shape 138 and cone shape 140. Fastener 142 holds inflatable bag 132 to cone shape 140. Fastener 142 may be, for example, a zipper, snaps, hook and loop, a belt, etc. When fastener 142 is unfastened, inflatable bag 132 has flat shape 138. When fastener 142 is fastened, inflatable bag 132 forms cone shape 140. Cone shape 140 is sized and shaped to conform to the shape of breast 106. Fastener 142 is adjustable such that cone shape 140 can be sized to fit the breast size and shape of different users.

Inflatable bag 132 includes chambers 144. Chambers 144 fill with air when inflatable bag 132 is inflated. Inflated chambers 144 provide compression pressure on breast 106. Stitching 146 creates and delineates chambers 144 of inflatable bag 132. Stitching 146 may be in a variety of patterns to create chambers 144 such as spiral 148. Chambers 144 may include rigid ribs to assist in applying compression pressure. Compression device 112 may use rigid ribs to apply compression pressure in the absence of inflatable bag 132.

Compression device 112, including inflatable bag 132, includes orifice 150. Orifice 150 provides a channel for trunk 122 to extend through for connection to shield 114. When shield 114 is connected to nipple cone 110, compression device 112 is positioned between shield 114 and nipple cone 110 as trunk 122 of nipple cone 110 extends through orifice 150 of compression device 112.

As depicted, housing 116 of lactation aid apparatus 102 can comprise a number of different components. For example, housing 116 can comprise pump 152, controller 154, display 156, user controls 158, battery 160, charge port 162, and wireless transceiver 164.

Pump 152 may be a set of pumps including suction pump 170 and air pump 172. As used herein, a “set of,” when used with reference to items, means one or more items.

Suction pump 170 includes first pressure valve 174 and air pump 172 includes second pressure valve 176. In this illustrated example, suction pump 170 is in fluid communication with collection container 118 where collection container 118 is positioned between shield 114 and suction pump 170. First pressure valve 174 is in fluid communication with collection container 118 and first pressure valve 174 is positioned between collection container 118 and suction pump 170. Further, in this illustrated example, second pressure valve 176 is in fluid communication with inflatable bag 132 and air pump 172. Second pressure valve 176 is positioned between inflatable bag 132 and air pump 172.

User 104 may operate lactation aid apparatus 102 through user controls 158 of housing 116. Example of user controls 158 include power on/off, adjusting pump pressure, programming use schedules, etc. Display 156, for example, may be an LED or LCD. Display 156 may indicate the current operating status of lactation aid apparatus 102 and, for example, can display statistics of uses including date, time, duration, and volume collected during use. Housing 116 contains battery 160 which provides power for operation of lactation aid apparatus 102. Battery 160 may be, for example, a replaceable battery, an interchangeable rechargeable battery, or a rechargeable battery that can be connected to a recharging power source through charge port 162. In other words, lactation aid apparatus 102 is not required to be plugged into a wall power outlet during operation. Wireless transceiver 164 may communicate with an application resident on a user's mobile device through wireless transmissions including wi-fi and Bluetooth. Lactation aid apparatus 102 can be operated through the application on the mobile device or through user controls 158 included in housing 116.

Lactation aid apparatus 102 is wearable. In other words, all components of lactation aid apparatus 102 are portable and can be concealed under the user's clothing. Nipple cone 110, compression device 112, and shield 114 can be worn under the user's bra. The user's bra helps secure nipple cone 110, compression device 112, and shield 114 and keep those components properly positioned during use. Housing 116 may be strapped to the user's body with a clip, belt, or strap or carried in a pouch and covered with the user's clothing.

In this illustrative example, operation of lactation aid apparatus 102, either through an application on a mobile device or user controls 158, is controlled by controller 154. Controller 154 can control the operation of suction pump 170, air pump 172, first pressure valve 174, and second pressure valve 176.

Controller 154 can be implemented in software, hardware, firmware, or a combination thereof. When software is used, the operations performed by controller 154 can be implemented in program code configured to run on hardware, such as a processor unit. When firmware is used, the operations performed by controller 154 can be implemented in program code and data and stored in persistent memory to run on a processor unit. When hardware is employed, the hardware can include circuits that operate to perform the operations in controller 154.

In the illustrative examples, the hardware can take a form selected from at least one of a circuit system, an integrated circuit, an application specific integrated circuit (ASIC), a programmable logic device, or some other suitable type of hardware configured to perform a number of operations. With a programmable logic device, the device can be configured to perform the number of operations. The device can be reconfigured at a later time or can be permanently configured to perform the number of operations. Programmable logic devices include, for example, a programmable logic array, a programmable array logic, a field programmable logic array, a field programmable gate array, and other suitable hardware devices. Additionally, the processes can be implemented in organic components integrated with inorganic components and can be comprised entirely of organic components excluding a human being. For example, the processes can be implemented as circuits in organic semiconductors.

In the illustrative example, controller 154 can control pump 152 to move air to provide a vacuum within collection container 118 in order to draw in expressed breast milk from reservoir 124 into collection container 118. Controller 154 can control pump 152 to move air to inflate and deflate inflatable bag 132 in order to provide compression pressure on breast 106.

Controller 154 can control other operations of lactation aid apparatus 102 such as applying heat to compression device 112 so that the engagement of compression device 112 and user 104 is comfortable. Further, controller 154 can control other operations of lactation aid apparatus 102 such as running a sanitation cycle through nipple cone 110, reservoir 124, tubing 128, and collection container 118. Controller 154 can be programmed to automatically operate lactation aid apparatus 102 according to a programmable schedule. Controller 154 can be programmed to monitor the volume of expressed breast milk collected and automatically cease operation when a programmed threshold is reached.

Housing 116 is connected to collection container 118. Backflow prevention device 166 seals the connection between housing 116 and collection container 118. Backflow prevention device 166 prevents expressed breast milk collected in collection container 118 from interacting with the components housed within housing 116. Collection container 118 is graduated 168. In other words, collection container 118 includes markings that indicate the volume of breast milk collected therein. As collection container 118 will be exposed to collected breast milk, collection container 118 is comprised of glass or medical grade silicon or plastic.

In use, controller 154 operates compression device 112 to repeatedly apply and release compression pressure on the breast of the user to stimulate milk expression. The expressed breast milk is temporarily pooled in reservoir 124. Subsequently or simultaneously, controller 154 operates pump 152 to create a vacuum in collection container 118. Collection container 118 is connected to reservoir 124 with tubing 128. The vacuum in collection container 118 draws the expressed milk pooled in reservoir 124 through tubing 128 and into collection container 118 to be collected. The suction pressure created by the vacuum is not enough to stimulate milk expression. The suction pressure created by the vacuum in collection container 118 is only enough to draw the expressed breast milk from reservoir 124 to collection container 118. The suction pressure is not uncomfortable to the user and is generally undetectable by the user. Controller 154 can vary operation of pump 152 to vary the suction pressure accordingly. Controller 154 can record duration and volume of milk collected during each use.

As used herein, a first component “connected to” or “coupled to” or “associated with” a second component means that the first component can be connected directly or indirectly to the second component. The connection is a physical association. In other words, additional components may be present between the first component and the second component. The first component is considered to be indirectly connected to the second component when one or more additional components are present between the two components. When the first component is directly connected to the second component, no additional components are present between the two components.

For example, a first component can be considered to be physically connected to a second component by at least one of being secured to the second component, bonded to the second component, mounted to the second component, welded to the second component, fastened to the second component, or connected to the second component in some other suitable manner. The first component also can be connected to the second component using a third component. The first component can also be considered to be physically connected to the second component by being formed as part of the second component, an extension of the second component, or both.

As used herein, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C or item B and item C. Of course, any combinations of these items can be present. In some illustrative examples, “at least one of” can be, for example, without limitation, two of item A; one of item B; and ten of item C; four of item B and seven of item C; or other suitable combinations.

The illustration of lactation collection environment 100 in FIG. 1 is not meant to imply physical or architectural limitations to the manner in which an illustrative embodiment may be implemented. Other components in addition to or in place of the ones illustrated may be used. Some components may be unnecessary. Also, the blocks are presented to illustrate some functional components. One or more of these blocks may be combined, divided, or combined and divided into different blocks when implemented in an illustrative embodiment.

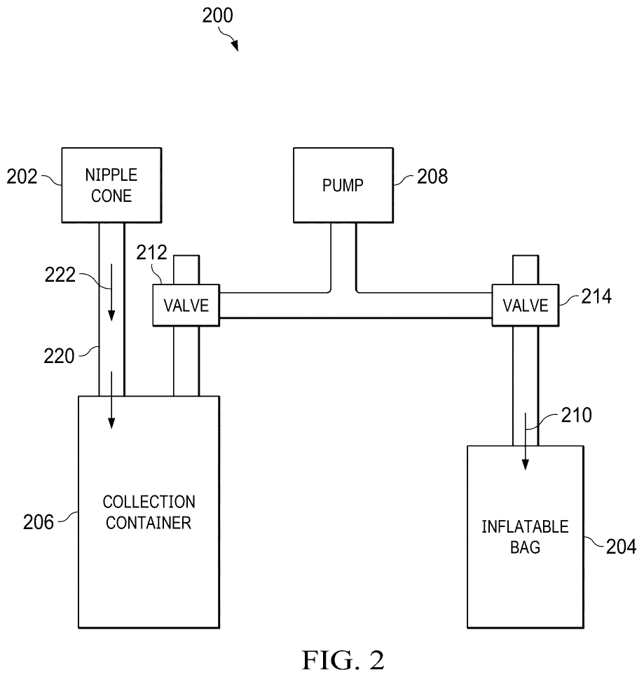

With reference next to FIG. 2, an illustration of a block diagram of a lactation aid apparatus is depicted in accordance with an illustrative embodiment. In this illustrative example, lactation aid apparatus 200 is an example of one implementation for lactation aid apparatus 102 shown in block form in FIG. 1.

In this illustrative example, lactation aid apparatus 200 includes nipple cone 202, inflatable bag 204, collection container 206, pump 208, first valve 212, and second valve 214.

Inflatable bag 204 is fitted to and positioned on a breast of a lactating user. Pump 208 and valve 214 operate to move air 210 to repeatedly inflate and deflate inflatable bag 204. The repeated inflation and deflation of inflatable bag 204 applies and releases compression pressure on the breast of the lactating user. The compression pressure stimulates the breast to express milk. The expressed milk pools within nipple cone 202 and a shield connected to nipple cone 202.

Nipple cone 110 flexibly engages the breast of the lactating user. Tubing 220 fluidly connects nipple cone 202 to collection container 206 and provides a path for expressed milk 222 from nipple cone 202 to collection container 206. Pump 208 and valve 212 operate to move air to create a vacuum within collection container 206. The vacuum in collection container 206 draws expressed milk 222 pooled in a reservoir formed by nipple cone and a breast shield through tubing 220 and into collection container 206 to be collected. Tubing 220 is comprised of medical grade silicone tubing.

In this illustrated example, pump 208 is in fluid communication with collection container 206 where collection container 206 is positioned between a breast shield that is connected to nipple cone 202 and pump 208. Valve 212 is in fluid communication with collection container 206. Valve 212 is positioned between collection container 206 and pump 208. Further, in this illustrated example, valve 214 is in fluid communication with inflatable bag 204 and pump 208. Valve 214 is positioned between inflatable bag 204 and pump 208.

In this illustrated example, pump 208 may comprise two separate pumps, an air pump for inflating and deflating inflatable bag 204 and a suction pump for creating a vacuum in collection container 206 to draw expressed milk that is pooled in nipple cone 202 into collection container 206.

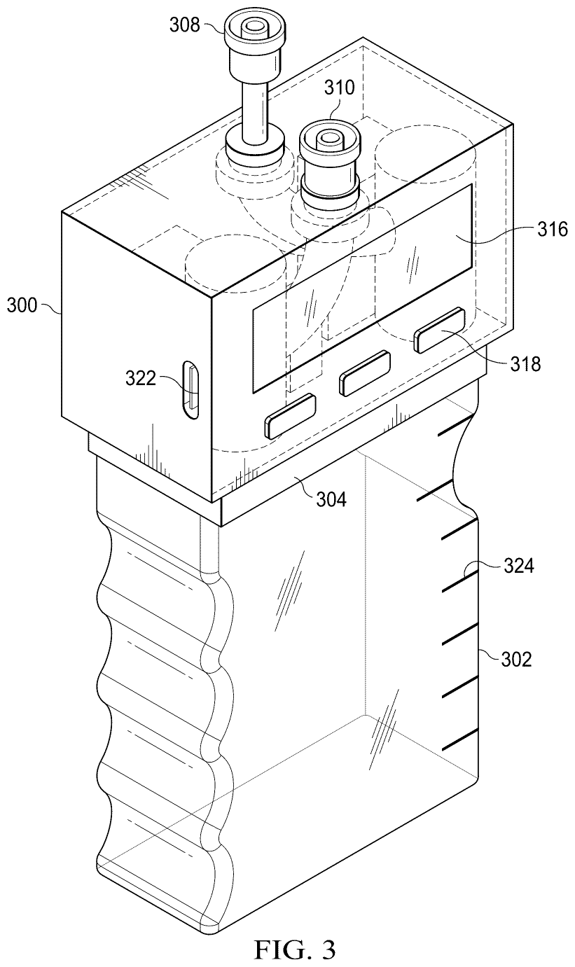

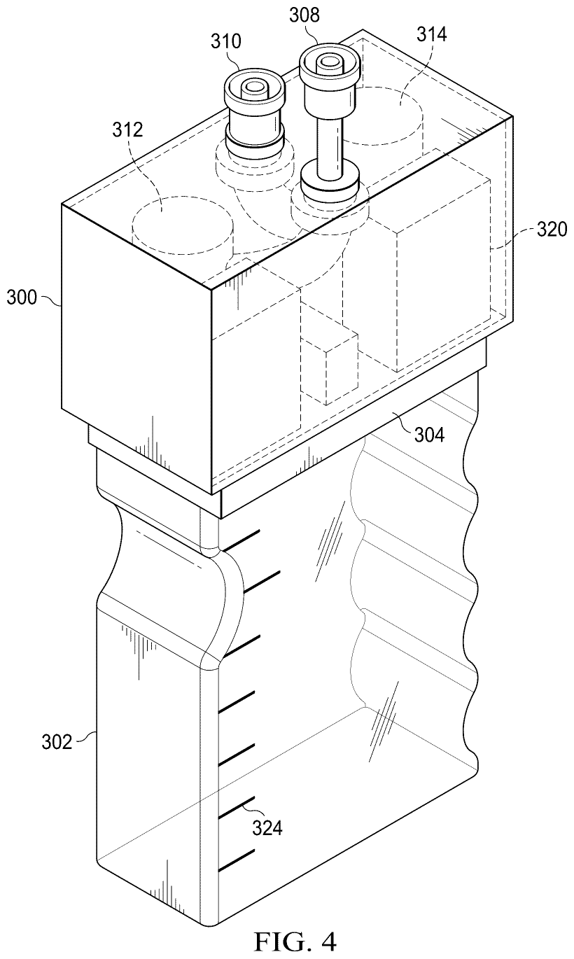

Turning now to FIGS. 3-4, illustrations of components of a housing and collection container of a lactation aid apparatus are depicted in accordance with an illustrative embodiment. In the illustrative examples, the same reference numeral may be used in more than one figure. This reuse of a reference numeral in different figures represents the same element in the different figures. The components illustrated in FIGS. 3-4 are examples of components that can be found in lactation aid apparatus 102 in FIG. 1 and lactation aid apparatus 200 in FIG. 2. As depicted, these components include at least housing 300 and collection container 302.

As depicted, housing 300 houses components of the lactation aid apparatus. As depicted, housing 300 is generally rectangular, however, other shapes as necessary for comfort or portability are envisioned without compromising the function of housing 300. Housing 300 is connected to collection container 302. Backflow prevention device 304 seals the connection between housing 300 and collection container 302. Backflow prevention device 304 prevents collected breast milk contained in collection container 302 from backflowing or spilling into housing 300 and prevents fluid damage to the components of lactation aid apparatus housed in housing 300.

Housing 300 contains air port 308, vacuum port 310, air pump 312, and suction pump 314. Air port 308 is connected to a compression device such as an inflatable bag. Air pump 312 is connected to air port 308 with internal tubing. Vacuum port 310 is connected to tubing that is connected to a nipple cone and a shield of the lactation aid apparatus. Suction pump 314 is connected to vacuum port 310 with internal tubing.

Housing 300 includes display 316. Display 316 is a readout where the user can get feedback on usage of the lactation aid apparatus. Housing includes user controls 318. A user can interact with and program the lactation aid apparatus manually through the use of user controls 318. A user may also interact with and program the lactation aid apparatus through an application running on a mobile device. Housing 300 includes battery 320. Battery provides electrical power to the components of the lactation aid apparatus that require it. Battery 320 may be replaceable or rechargeable. Housing 300 includes charge port 322 to recharge battery 320 when needed. Collection container 302 includes graduated markings 324. Graduated markings 324 indicate the volume of breast milk collected in collection container 302.

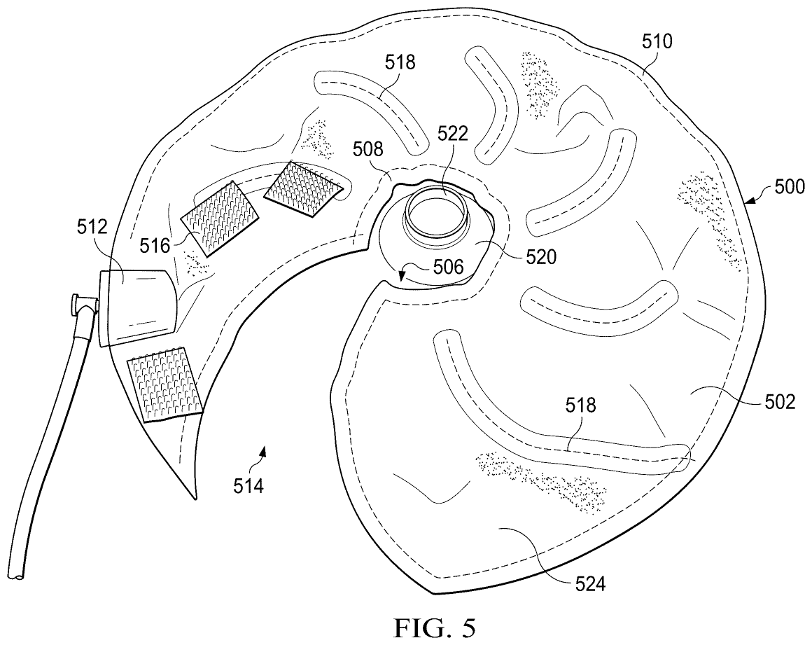

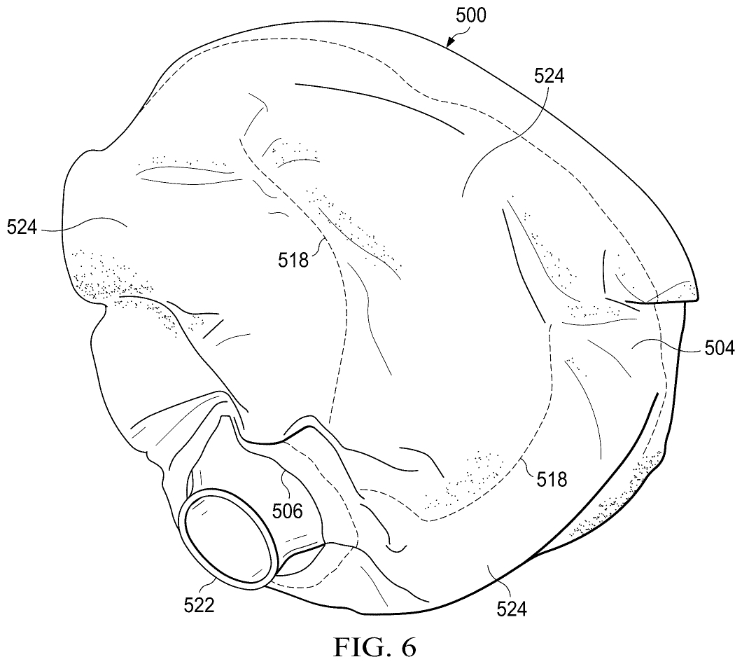

With reference now to FIGS. 5-6, illustrations of an inflatable bag of a lactation aid apparatus is depicted in accordance with an illustrative embodiment. Inflatable bag 500 is an example of one implementation for inflatable bag 132 in FIG. 1. Inflatable bag 500 can also be an example implementation for inflatable bag 204 in FIG. 2.

FIG. 5 depicts inflatable bag 500 in flat shape 502. Accordingly, FIG. 6 depicts inflatable bag 500 in cone shape 504.

As shown in FIGS. 5-6, inflatable bag 500 includes orifice 506. Orifice 506 is an opening through the center of inflatable bag 500. Trunk 522 of nipple cone 520 extends through orifice 506. Inner edge 508 is sealed and forms orifice 506. Outer edge 510 is sealed. Because inner edge 508 and outer edge 510 are sealed airtight, inflatable bag is capable of receiving air at air input port 512 to become inflated. Gap 514 is an interruption of the circumference of inflatable bag 500. Gap 514 extends from inner edge 508 to outer edge 510. Fastener 516 is positioned on inflatable bag 500 near gap 514. Fastener 516 is used to close gap 514 and transform inflatable bag 500 from flat shape 502 to cone shape 504. Fastener is adjustable to accommodate different sizes and shapes of different users. Inflatable bag 500 includes stitching 518. Stitching 518 segments inflatable bag 500 into chambers 524. Chambers 524 are inflatable pockets of inflatable bag 500. Repeatedly inflating and deflating inflatable bag 500 inflates and deflates chambers 524. As a result, inflating and deflating chambers 524 applies and releases compression pressure on the breast of the user.

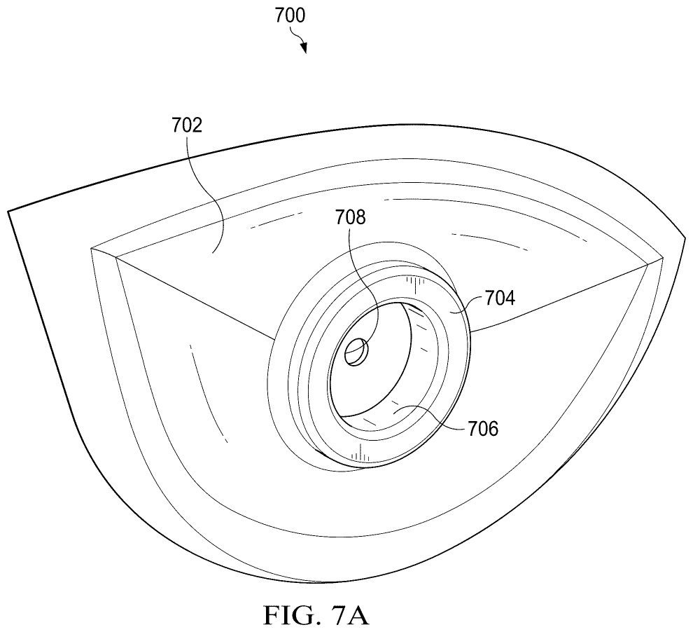

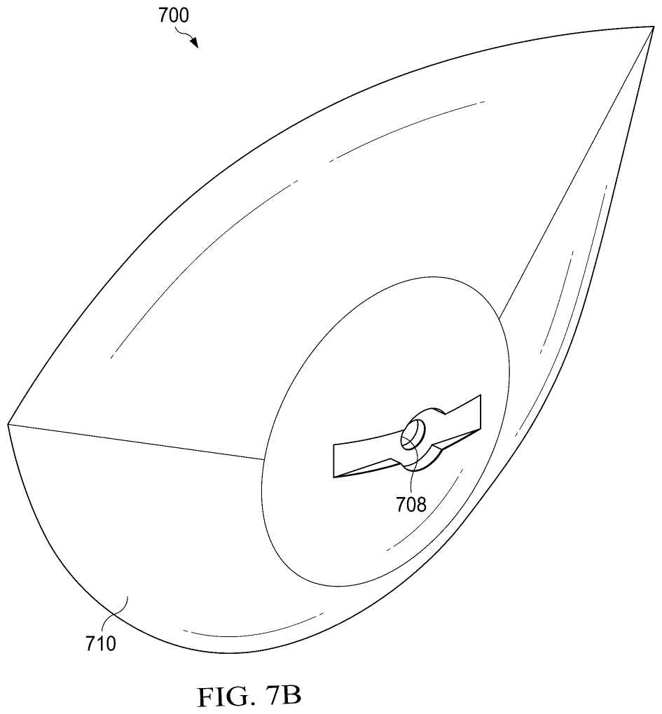

With reference now to FIGS. 7A-7B, illustrations of a shield of a lactation aid apparatus is depicted in accordance with an illustrative embodiment. Shield 700 is an example of one implementation for shield 114 in FIG. 1.

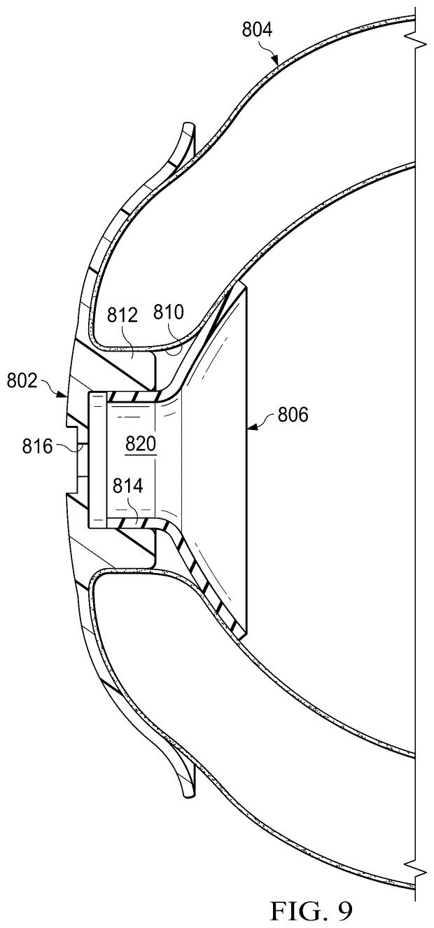

Shield 700 is connected to a nipple cone, such as nipple cone 520 of FIGS. 5-6. Shield 700 is generally cone shaped to conform to the natural shape of a user's breast. Concave side 702 of shield 700 includes internal shoulder 704 that friction fits with a nipple cone, for example, trunk 522 of FIGS. 5-6. Shield 114 is snapped onto and off of a nipple cone as normal use requires. Reservoir 706 is formed when a nipple cone is connected to shield 700 (discussed further in reference to FIG. 9). In use, breast milk expressed from a user will pool in reservoir 706 before the expressed breast milk is collected in collection container 118 with suction. In other words, reservoir 706 provides an initial collection point for expressed breast milk. Shield 700 includes channel 708. Channel 708 extends through shield 700 from reservoir 706 on concave side 702 to convex side 710. Channel 708 is connected to a collection container with tubing.

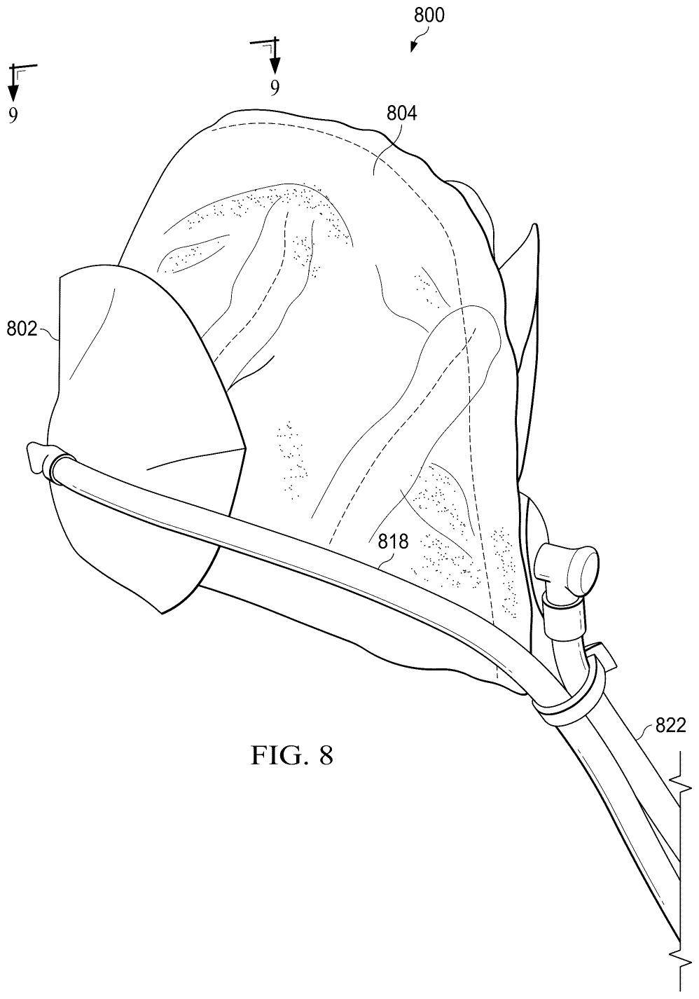

With reference to FIGS. 8-9, an illustration of a shield connected to a nipple cone and inflatable bag of a lactation aid apparatus is depicted in accordance with an illustrative embodiment. Lactation aid apparatus 800 is an example of one implementation for lactation aid apparatus 102 in FIG. 1 and lactation aid apparatus 200 in FIG. 2. Shield 802 is an example of one implementation for shield 114 in FIG. 1 and shield 700 in FIGS. 7A-7B. Inflatable bag 804 is an example of one implementation for inflatable bag 132 in FIG. 1, inflatable bag 204 in FIG. 2, and inflatable bag 500 in FIGS. 5-6. Nipple cone 806 is an example of one implementation for nipple cone 110 in FIG. 1, nipple cone 202 in FIG. 2, and nipple cone 520 in FIGS. 5-6.

In FIG. 9, a cross-sectional view of shield 802, nipple cone 806, and inflatable bag 804 of lactation aid apparatus 800 is shown taken along lines 9-9 in FIG. 8.

Inflatable bag 804 includes orifice 810. Trunk 814 of nipple cone 806 extends through orifice 810 for connection to interior shoulder 812 shield 802. When shield 802 is connected to nipple cone 806, inflatable bag 804 is positioned between shield 802 and nipple cone 806 as trunk 814 extends through orifice 810.

Reservoir 820 is formed when nipple cone 806 is connected to shield 802 and nipple cone 806 is fitted to a user. When using lactation aid apparatus 800, breast milk expressed from a user pools in reservoir 820 before the expressed breast milk is drawn to and collected in a collection container by a vacuum created in the collection container. Reservoir 820 provides an initial collection point for expressed breast milk. Shield 802 includes channel 816. Channel 816 extends through shield 802. Channel 816 is connected to a collection container with tubing 818. Tubing 818 is comprised of medical grade silicone tubing. Channel 816 and tubing 818 provide fluid communication between reservoir 820 and a collection container.

Air tubing 822 is connected to inflatable bag 804. Air tubing 822 is also connected to a pump, such as air pump 172 of FIG. 1 and pump 208 of FIG. 2. Air tubing 822 move air provided by a pump to repeated inflate and deflate inflatable bag 804. The repeated inflation and deflation of inflatable bag 804 provides compression pressure on the breast of a user which stimulates breast milk expression. Expressed breast milk resulting from the compression pressure pools in reservoir 820. A vacuum created in a collection container with a suction pump in fluid communication with the collection container draws the expressed milk pooled in reservoir 820 from reservoir 820 to the collection container.

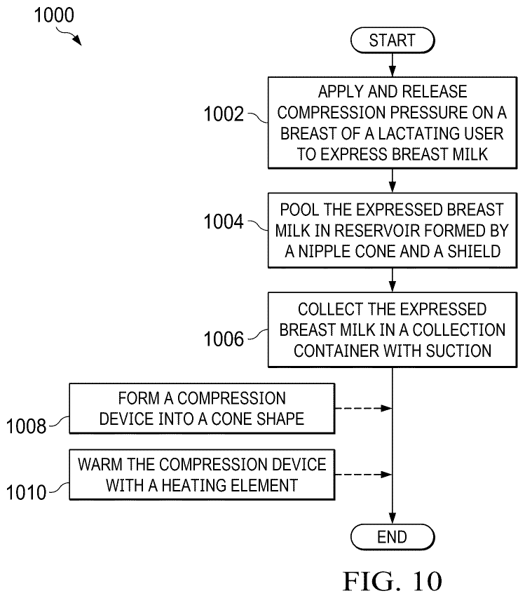

Turning next to FIG. 10, an illustration of a flowchart of a process 1000 for expressing and collecting breast milk is depicted in accordance with an illustrative embodiment.

The process begins by applying and releasing compression pressure on a breast of a lactating user (operation 1002). The compression pressure is applied with a compression device positioned on the breast of the user. The compression pressure stimulates the expression of breast milk. At operation 1004, the expressed breast milk is pooled in a reservoir formed by a nipple cone connected to a shield. The process continues by collecting the expressed breast milk in a collection container (operation 1006). The expressed breast milk is collected by applying suction to the shield connected to the compression device.

At operation 1008, the process forms the inflatable bag into a cone shape with a fastener connected to the inflatable bag. At operation 1010, the process warms the compression device with a heating element connected to the inflatable bag.

The flowcharts and block diagrams in the different depicted embodiments illustrate the architecture, functionality, and operation of some possible implementations of apparatuses and methods in an illustrative embodiment.

In some alternative implementations of an illustrative embodiment, the function or functions noted in the blocks may occur out of the order noted in the figures. For example, in some cases, two blocks shown in succession may be performed substantially concurrently, or the blocks may sometimes be performed in the reverse order, depending upon the functionality involved. Also, other blocks may be added in addition to the illustrated blocks in a flowchart or block diagram.

The description of the different illustrative embodiments has been presented for purposes of illustration and description and is not intended to be exhaustive or limited to the embodiments in the form disclosed. The different illustrative examples describe components that perform actions or operations. In an illustrative embodiment, a component can be configured to perform the action or operation described. For example, the component can have a configuration or design for a structure that provides the component an ability to perform the action or operation that is described in the illustrative examples as being performed by the component. Further, to the extent that terms “includes”, “including”, “has”, “contains”, and variants thereof are used herein, such terms are intended to be inclusive in a manner similar to the term “comprises” as an open transition word without precluding any additional or other elements.

Many modifications and variations will be apparent to those of ordinary skill in the art. Further, different illustrative embodiments may provide different features as compared to other desirable embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

What is claimed is:

1. A wearable lactation aid apparatus, comprising:
a nipple cone;
a compression device; and
a shield connected to the nipple cone, wherein the compression device is positioned between the shield and the nipple cone.

2. The apparatus of claim 1, further comprising:
a suction pump connected to the shield and in fluid communication with the nipple cone; and
a collection container in fluid communication with the suction pump and positioned between the shield and the suction pump.

3. The apparatus of claim 1, wherein the compression device is an inflatable bag, the apparatus further comprising an air pump connected to the inflatable bag.

4. The apparatus of claim 1, wherein the compression device is an inflatable bag, the apparatus further comprising:
a pump connected to the shield and to the inflatable bag;
a collection container in fluid communication with the pump and positioned between the shield and the pump;
a first pressure valve in fluid communication with the collection container and the pump and positioned between the collection container and the pump; and
a second pressure valve in fluid communication with the inflatable bag and the pump and positioned between the inflatable bag and the pump.

5. The apparatus of claim 2, wherein the shield and the nipple cone form a reservoir in fluid communication with the collection container.

6. The apparatus of claim 3, wherein the inflatable bag comprises stitching forming a plurality of chambers within the inflatable bag.

7. The apparatus of claim 3, wherein the inflatable bag includes a hook and loop fastener configured to fix the inflatable bag into a cone shape from a flat shape.

8. The apparatus of claim 1, wherein the nipple cone includes a trunk extending through an orifice in the compression device, the trunk connected to the shield.

9. The apparatus of claim 1, further comprising a heating element removably connected to the compression device.

10. The apparatus of claim 3, wherein the nipple cone comprises a trunk extending from a flexible flange, wherein the inflatable bag comprises an outer surface opposite a user facing inner surface, and wherein the flexible flange abuts the inner surface and the shield abuts the outer surface.

11. A system for expressing and collecting breast milk from a lactating user, comprising:
an inflatable bag positioned on a breast of the user;
a pump connected to the inflatable bag and configured to repeatedly inflate and deflate the inflatable bag to apply and release compression on the breast;
a nipple cone positioned on the breast of the user and extending through the inflatable bag for connection to a shield; and
the pump connected to the shield and in fluid communication with a collection container positioned between the pump and the shield.

12. The system of claim 11, wherein the shield and the nipple cone form a reservoir in fluid communication with the collection container.

13. The system of claim 11, wherein the pump comprises a suction pump connected to the shield and in fluid communication with the nipple cone and an air pump connected to the inflatable bag.

14. The system of claim 13, further comprising a first pressure valve in fluid communication with the collection container and the pump and positioned between the collection container and the pump and a second pressure valve in fluid communication with the inflatable bag and the pump and positioned between the inflatable bag and the pump.

15. The system of claim 11, wherein the inflatable bag comprises stitching in a spiral pattern, the stitching forming a plurality of chambers within the inflatable bag.

16. The system of claim 11, further comprising a backflow prevention device connected to the pump and the collection container.

17. A method for expressing and collecting breast milk, the method comprising:
applying and releasing compression pressure with a compression device positioned on a breast of a lactating user to express breast milk; and
collecting the expressed breast milk in a collection container with suction applied to a shield connected to the compression device.

18. The method of claim 17, further comprising:
pooling the expressed breast milk in a reservoir formed by a nipple cone and the shield, the nipple cone extending through the compression device and connected to the shield.

19. The method of claim 17, further comprising:
warming the compression device with a heating element connected to the compression device.

20. The method of claim 17, wherein the compression device is an inflatable bag, the method further comprising:
forming the inflatable bag into a cone shape with a fastener connected to the inflatable bag.
